(19) United States
(12) Patent Application Publication
(10) Pub. No.: US 2003/0206902 A1
(43) Pub. Date: Nov. 6, 2003

(54) Ve-cadherin and vegfr-2 antagonists for therapeutic modulation of angiogenesis

(76) Inventors: Fang Liao, New York, NY (US); Daniel Hicklin, Upper Montclair, NJ (US); Peter Boehlen, New York, NY (US)

Correspondence Address: KENYON & KENYON, ONE BROADWAY, NEW YORK, NY, 10004, US

(21) Appl. No.: 10/220,831

(22) PCT Filed: Mar. 5, 2001
(86) PCT No.: PCT/US01/06966

(51) Int. Cl. A61K 48/00; A61K 38/17; A61K 39/395

(52) U.S. Cl. 424/143.1; 514/12; 514/44

(57) ABSTRACT

The present invention includes the use of an antagonist of vascular endothelial growth factor receptor (VEGFR) in combination with an antagonist of vascular endothelial cadherin (VE-cadherin) to modulate angiogenesis. Included is the use of the VEGFR antagonist and the antagonist of VE-cadherin in a manner so as to prevent or ameliorate toxicity associated with the use of VE-cadherin antagonist. The present invention also comprises the use of a VEGFR antagonist and a VE-cadherin antagonist for treating, with reduced toxicity, diseases associated with angiogenesis, which include but are not limited to neoplastic diseases, autoimmune diseases and collagen vascular diseases, as well as the use of a VEGFR antagonist for the treatment of disease states associated with pathological vascular permeability which include, but are not limited to, ARDS, edema states and related conditions. Also included are compositions comprising a VEGFR antagonist, or a VEGFR antagonist and a VE-cadherin antagonist for treating such conditions.

This application claims the benefit of U.S. Provisional Application No. 60/187,218 filed on Mar. 3, 2000.

### FIELD OF THE INVENTION

The present invention relates to the use of an antagonist of vascular endothelial growth factor receptor (VEGFR) in combination with an antagonist of vascular endothelial cadherin (VE-cadherin) to modulate angiogenesis and to prevent or ameliorate toxicity associated with the use of a VE-cadherin antagonist. The present invention also comprises a method of treating, with reduced toxicity, diseases associated with angiogenesis, and treating disease states associated with pathological vascular permeability.

### BACKGROUND OF THE INVENTION

Many disease states, comprising a wide range of disease types, are associated with an abnormal proliferation of blood vessels. Examples of such disease states include, but are not limited to, neoplastic diseases, including but not limited to solid tumors, autoimmune diseases and collagen vascular diseases, including but not limited to rheumatoid arthritis, and age-related macular degeneration. One means of controlling such diseases comprises restricting the blood supply to those cells involved in mediating or causing the disease. Such diseases are dependant, at least in part, upon the development of new blood vessels at the sites of disease necessary for the disease-causing cells to survive and/or proliferate. By way of illustration which is not intended to be limiting in any way, once solid tumors develop to a size of about a few millimeters, further growth is not possible absent neovascularization, or new vessel formation—the development of new blood vessels, within the tumor. In the past, strategies to limit the blood supply to tumors have included occluding blood vessels supplying portions of organs in which tumors are present. Such approaches require the site of the tumor to be identified and are generally limited to treatment to a single site or to a small number of sites. A better method of treatment is administering, systemically or regionally, agents to restrict or eliminate the blood supply to the cells responsible for the disease, while limiting or avoiding harm to normal surrounding tissue. This would not require identification and/or precise localization of disease sites. Such an approach also would permit the control of diseases which are widespread, or the sites of which are too numerous or too small to restrict but would also be of value in controlling larger tumors, preventing their further growth by preventing the development of new blood vessels to support their further growth. For example, a tumor's blood supply could be restricted in a more direct and limited manner and the growth of the tumor could be controlled or prevented.

Angiogenesis is a highly complex process that involves the proliferation and migration of endothelial cells, the capacity of endothelial cells to invade tissues, cell assembly into tubular structures, joining of newly forming tubular assemblies to closed-circuit vascular systems, and maturation of newly formed capillary vessels. The molecular bases of many of these aspects are still not understood. Because of the important role that excessive angiogenesis plays in the pathogenesis of a variety of diseases, including the growth of neoplasms, including solid tumors, and autoimmune and collagen vascular diseases such as rheumatoid arthritis and age-related macular degeneration, there is much interest in the development of ways to inhibit or prevent angiogenesis in these settings.

Ideally, such a means of inhibiting angiogenesis will interfere with the angiogenesis or neovascularization associated with these conditions without interfering with the normal vasculature of other organs or tissues. A limited understanding of the biology of angiogenesis and neovascularization has hampered the identification of suitable strategies to achieve this goal.

One target of interest is the cadherins, a rapidly increasing family of cell adhesion molecules involved in the formation of specific cell-cell contacts ((Takeichi, Ann. Rev. Biochem. 59: 237-252 (1990); Geiger & Ayalon, Ann. Rev. Cell Biol. 8: 302-332 (1992); Uemura, Cell 93: 1095-1098 (1998). Cadherins are single chain transmembrane glycoproteins with molecular weights of about 120-140 kD. Members of this family exhibit calcium-dependent homophilic interactions and have a role in selective cell-cell recognition and adhesion, which is necessary for allocating different cell types to their proper places during development. Cadherins also play an important role in maintaining the integrity of multicellular structures. During embryonic morphogenesis the expression of diverse members of the cadherin family is spatially and temporally regulated, facilitating the orderly assembly of various cell types into functional structures.

Cadherins have typical structural features and share considerable sequence homology (43-58%). Their extracellular region typically contains 5 repeating domains of approximately 110 amino acids. The N-terminal domain has been shown to be important in homotypic cell-cell interaction as evidenced by experiments with molecular chimeras, monoclonal antibodies and peptide inhibitors, and the 3-dimensional structures of the N-terminal domains of N-cadherin and E-cadherin have been demonstrated. Accordingly, it is believed that cadherins form dimers supported by zipper-like elements and possibly by disulfide linkage. The short intracellular portion of cadherins is their most highly conserved region and plays a role in classic cadherin function by anchoring cadherins to the cytoskeleton and providing signaling functions through cadherin phosphorylation.

The events involving cytoplasmic clustering strengthen the extracellular domain-mediated cadherin adhesive activity. Among the cadherins, E-cadherin is best-characterized, loss or mutation of this cadherin has been associated with increased invasiveness and metastasis of certain human tumors.

Vascular endothelial cadherins (VE-cadherins) are endothelial specific cadherins strictly localized at intracellular junctions of essentially all types of endothelium. One endothelial-cell specific cadherin, VE-cadherin (or cadherin-5) has been shown to be localized at certain intercellular junctions—adherens junctions (AJ) in cell-to-cell contacts. A number of experimental observations suggest that this cadherin is involved in various aspects of vascular biology related to angiogenesis, including the assembly of endothelial cells into tubular structures. For example, thrombin-induced vascular permeability is shown to be associated with disassembly of endothelial adherents junctions, and VE-cadherin and its N-terminal fragment inhibit the density-dependent growth and migration of endothelial cells. VE-cadherin also confers adhesive properties to transfected cells.

VE-cadherin null mice have been developed and have been shown to have severe defects in the development of vascular structures, resulting in an embryonic lethal phenotype. All these data have led investigators to explore the cadherins, including VE-cadherin as potential targets to inhibit neovascularization. However, to date, attempts to inhibit neovascularization in test animals with the use of cadherin antagonists have been complicated by the development of syndromes in which there has been wide-spread vascular disruption involving the normal blood vessels or various organs including the lungs and heart, resulting in severe toxicity, including hemorrhage and death. Attempts to utilize lower doses of antagonists have resulted in the failure to prevent neovascularization. The present invention addresses such problems, while permitting the use of more than one agent to inhibit angiogenesis.

Vascular endothelial growth factor (VEGF) is a potent vascular permeability factor in vitro and in vivo, an endothelial cell-specific mitogen, and is a possible regulator of angiogenesis in vivo. However, the molecular mechanism by which VEGF mediates vascular permeability is obscure. Recent, experimental evidence based on in vitro studies suggests that such permeability effect is related to dissociation of AJ and tight junctions (TJ), presumably by modulating the phosphorylation states of several junctional molecules associated with AJ and TJ. As a major component of AJ, VE-cadheren is a potential target for VEGF.

VEGF is a homodimeric glycoprotein consisting of two 23 kD sub units. Four different monomeric isoforms of VEGF have been identified, resulting from alternative splicing of mRNA. These include two membrane bound forms (VEGF 206 and VEGF 189) and two soluble forms (VEGF 165 and VEGF 121). In all human tissues except placenta, VEGF 165 is the most abundant isoform. VEGF is expressed in embryonic tissues (Breier et al., Development (Camb.) 114:521 (1992)), macrophages, and proliferating epidermal keratinocytes during wound healing (Brown et al., J. Exp. Med., 176:1375 (1992)) and may be responsible for tissue edema associated with inflammation (Ferrara et al., Endocr. Rev. 13:18 (1992)). In situ hybridization studies have demonstrated high VEGF expression in a number of human tumor lines including glioblastoma multiform, hemangioblastoma, central nervous system neoplasms and AIDS-associated Kaposi's sarcoma (Plate, K. et al. (1992) Nature 359: 845-848; Plate, K. et al. (1993) Cancer Res. 53: 5822-5827; Berkman, R. et al. (1993) J. Clin. Invest. 91: 153-159; Nakamura, S. et al. (1992) AIDS Weekly, 13 (1)). High levels of VEGF were also observed in hypoxia induced angiogenesis (Shweiki, D. et al. (1992) Nature 359: 843-845).

The biological response of VEGF is mediated through its high affinity VEGF receptors which are selectively expressed on endothelial cells during embryogenesis (Millauer, B., et al. (1993) Cell 72: 835-846) and during tumor formation. VEGF receptors are characterized by having several, typically 5 or 7, immunoglobulin-like loops in their amino-terminal extracellular receptor ligand-binding domains (Kaipainen et al., J. Exp. Med. 178:2077-2088 (1993)). The other two regions include a transmembrane region and a carboxy-terminal intracellular catalytic domain interrupted by an insertion of hydrophilic interkinase sequences of variable lengths, called the kinase insert domain (Terman et al., Oncogene 6:1677-1683 (1991). VEGF receptors include FLT-1 (Shibuya M. et al., Oncogene 5, 519-524 (1990); Terman et al., Oncogene 6:1677-1683 (1991)) and FLK-1 (Matthews W. et al., Proc. Natl. Acad. Sci. USA, 88:9026-9030 (1991)).

Receptor activity is probably induced during tumor formation, and this upregulation is confined to the vascular endothelial cells in close proximity to the tumor. Blocking VEGF activity with neutralizing anti-VEGF monoclonal antibodies (mAbs) resulted in inhibition of the growth of human tumor xenografts in nude mice (Kim, K. et al., Nature 362: 841-844 (1993)), indicating a role for VEGF in tumor-related angiogenesis.

### BRIEF DESCRIPTION OF THE INVENTION

The present invention comprises a method of inhibiting pathologic angiogenesis while limiting or preventing toxicity by administering a VE-cadherin antagonist in combination with a VEGFR antagonist. Optionally the VE-cadherin antagonist can be administered with the VEGFR antagonist, after or before the VEGFR antagonist. Another embodiment of the present invention comprises a pharmaceutical composition comprising at least one antagonist of a vascular endothelial cadherin(VE-cadherin) peptide, and at least one antagonist of a vascular endothelial growth factor receptor (VEGFR). Suitable antagonists of VE-cadherin peptide include, but are not limited to proteins, peptides, polypeptides, antisense molecules, small molecules, antibodies, antibody fragments and polynucleotides.

The invention also provides for a method of treating, ameliorating, or preventing cell proliferative diseases by administering to subjects in need thereof a composition containing one or more antagonists of VE-cadherin peptide in combination with an antagonist of VEGFR. The invention also provides a method of inhibiting angiogenesis, or tumor growth in a mammal comprising administering the compositions of this invention.

The present invention comprises a method of inhibiting pathological angiogenesis in an animal in need thereof, comprising administering an antagonist of a vascular endothelial cadherin and administering an antagonist of a vascular endothelial growth factor receptor. Preferably the animal is a mammal, more preferably a human. Optionally, the VEGFR is FLK-1, KDR, FLT-1 or FLT-4, and in a preferred embodiment is KDR. The VEGFR antagonist optionally is a protein, a peptide, a polypeptide, an antisense molecule, a small molecule, an antibody, or an antibody fragment. The antagonist of vascular endothelial cadherin optionally is a protein, a peptide, a polypeptide, an antisense molecule, a small molecule, an antibody, or an antibody fragment. Preferred VE-cadherin antibodies include BV13 (previously known as 19E6), 10G4, BV6, BV4, Cad-5, BV9, TEA, and Hec 1.2, more preferably BV13. Optionally the VEGFR antagonist is p1C11 or DC101 mAb. Also encompassed by the present invention are methods of modulating angiogenesis and of treating, with reduced toxicity, a disease associated with pathological vascular permeability, in an animal in need thereof, while reducing toxicity, comprising administering an antagonist of VE-cadherin and administering an antagonist of VEGFR. Such diseases include, but are not limited to neoplastic diseases including solid tumors, autoimmune diseases and collagen vascular diseases. Optionally, the antagonists of VE-cadherin and VEGFR are administered at substantially the same time or one is administered from about a few minutes to a few hours prior to the other, preferably the VEGFR antagonist first. One preferred embodiment of the present invention comprises administering p1C11 and BV13 to modulate angiogenesis in an animal in need thereof, while reducing toxicity. Also encompass within the scope of the present invention is a composition comprising an antagonist of VEGFR and an antagonist of VE-cadherin. The scope of the present invention also encompasses a method of inhibiting pathologically increased vascular permeability to an animal in need thereof, comprising administering an antagonist of a VEGFR. In a preferred embodiment the antagonist the monoclonal antibody p1C11. Within the scope of the present invention, the VEGFR antagonist optionally can interact with the VEFR receptor itself or with its ligand.

### BRIEF DESCRIPTION OF THE FIGURES

FIG. 1: VE-cadherin Dimerization. Two forms of VE-cadherin dimers are proposed based on the crystal structures resolved for N-and E-cadherins. The “strand dimer” (left panels) refers to homophilic interactions between two VE-cadherin molecules on the surface of the same cell. The “adhesive dimer” (right panels) refers to homophilic interactions between VE-cadherin molecules located on opposing cells.

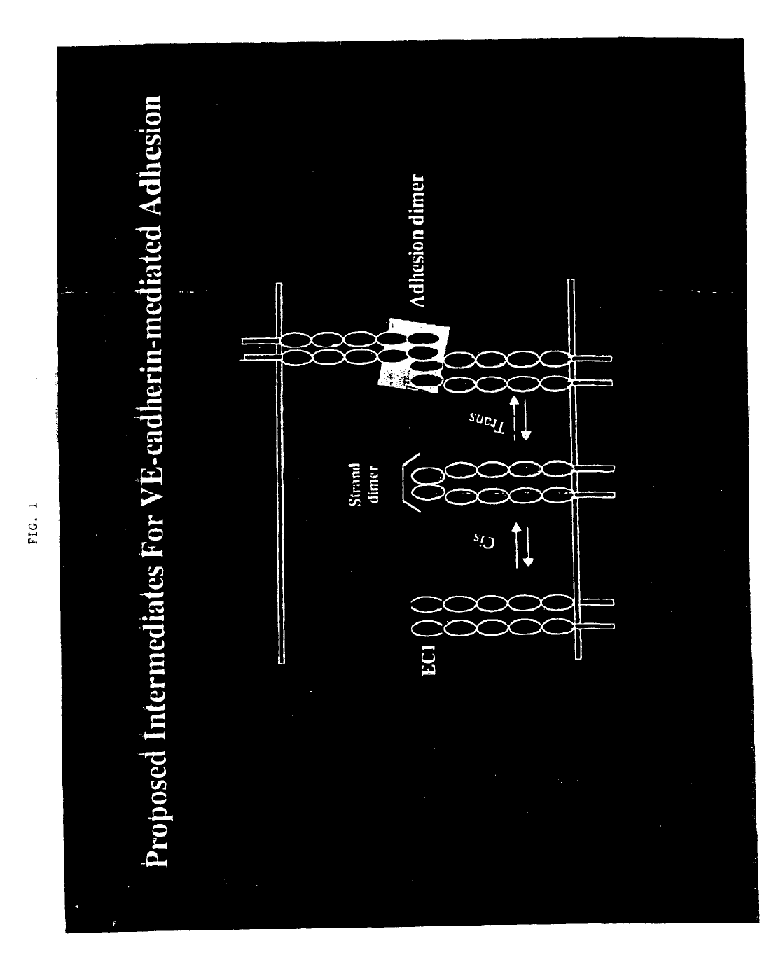

FIG. 2: Anti-VE-cadherin blocking antibody BV13 increases vascular permeability in vivo.

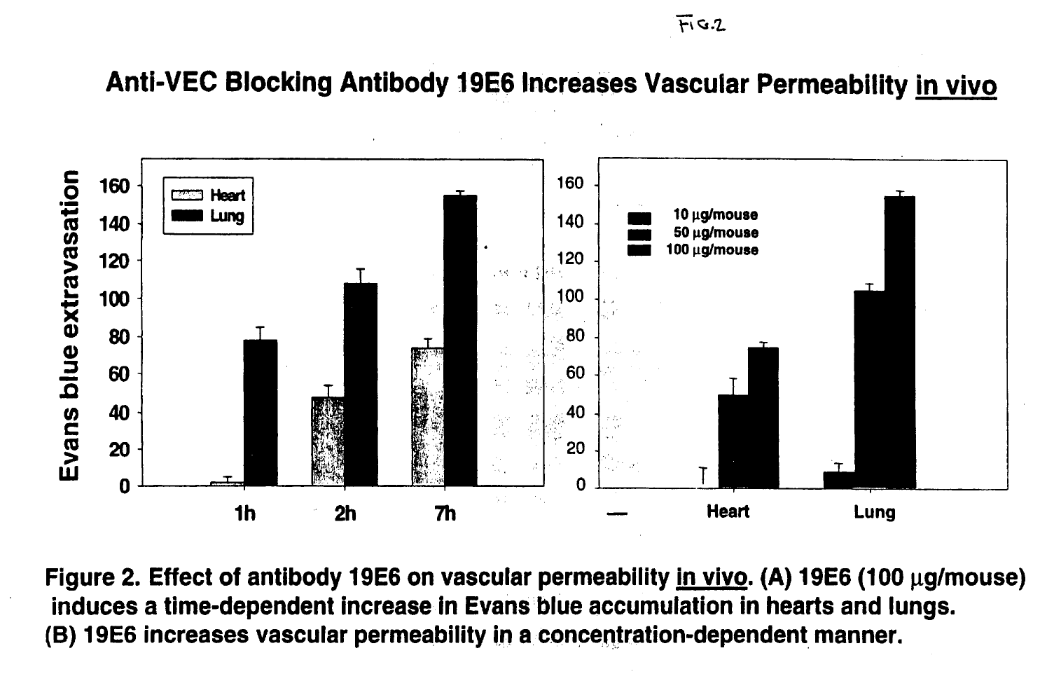

FIG. 3: Anti-VEGFR-2/FLK-1 antibody DC101 abolishes the VEGF-induced permeability effect.

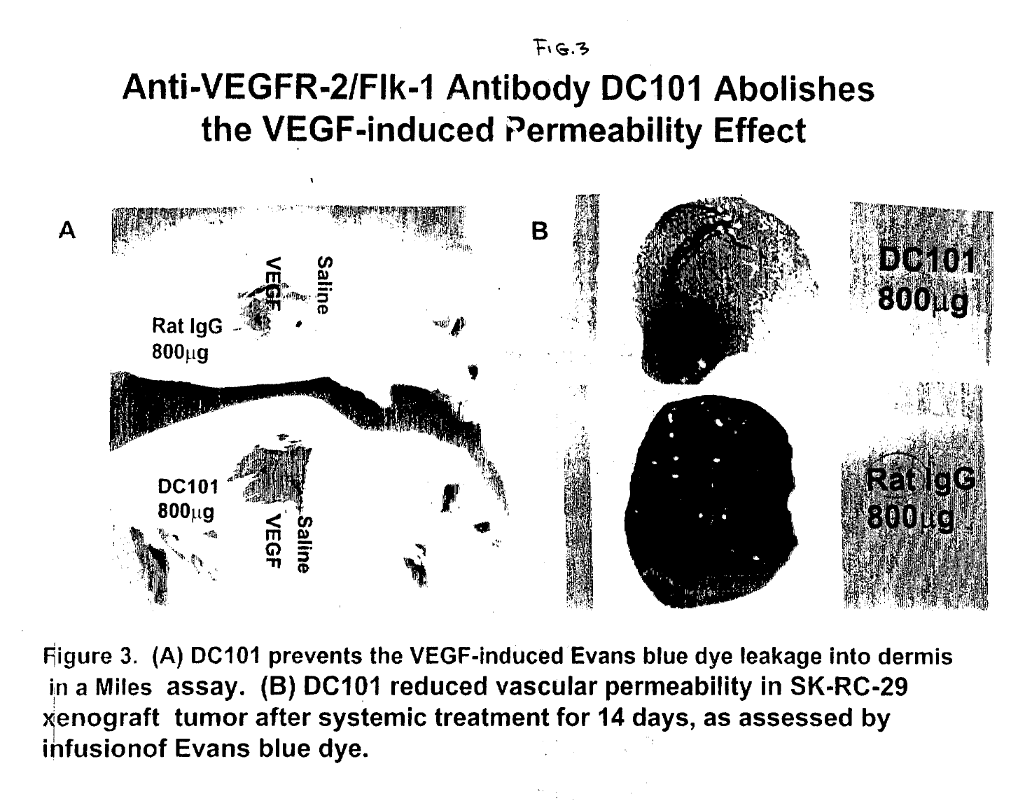

FIG. 4: DC101 rescues mice from BV13 induced lethality.

FIG. 5: Combination of mAb BV13 and DC101 results in negligible change in vascular permeability in mice.

FIG. 6: Anti VE-cadherin mAb BV13 inhibits tumor growth and metastasis.

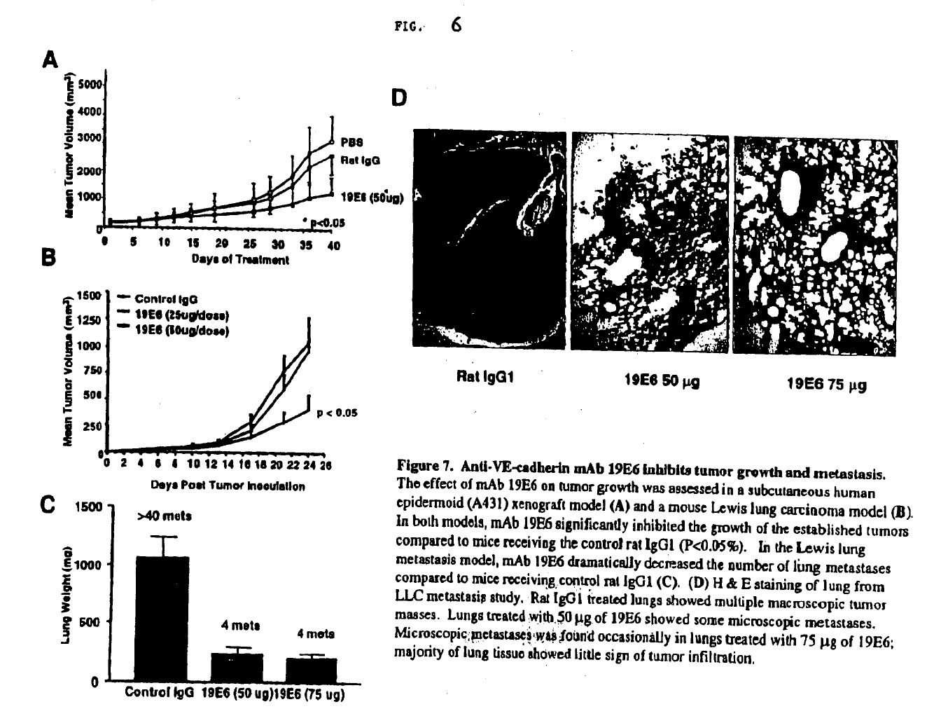

### DETAILED DESCRIPTION OF THE INVENTION

The present invention comprises the use of an VE-cadherin antagonist together with a VEGFR antagonist to overcome the toxicity, which can include increased vascular permeability, hemorrhage and even death, previously associated with the administration of therapeutic doses of VE-cadherin antagonist.

The “antagonist” of this invention includes any compound, composition or small molecule that interferes with or blocks the activity and/or expression of one or more VE-cadherin gene product(s) in vitro, ex vivo, or in vivo, or interferes with, inhibits, or blocks the binding of VEGF to its receptor and/or activation of VEGF receptors. The antagonists include, but are not limited to protein, peptide, polypeptide, or an antisense molecule, small molecules, antibodies, antibody fragments, and the like. The antagonists of the present invention comprise[s], for example, recombinant polypeptide, natural polypeptide or synthetic polypeptide. It is to be understood that the VEGF antagonist of the present invention optionally will target either the ligand VEGF or its receptor, such that the binding of VEGF to its receptor and/or activation of VEGF receptors is interfered with, inhibited, or blocked. Further, for the purposes of the present application, it is to be understood that the terms VEGFR antagonist or antagonist of vascular endothelial growth factor receptor are to be understood to include those antagonists that interact with or bind to either the VEGF ligand or the VEGF receptor (VEGFR).

Unexpectedly, the inventors of the present invention have found that when VEGFR antagonist is administered together with a therapeutic dose of VE-cadherin antagonist, the toxicity of the VE-cadherin antagonist is reduced, such that the increase in vascular permeability is reduced or eliminated, hemorrhage is diminished or absent and the risk of death is greatly reduced. In such a way the therapeutic index of VE-cadherin antagonists are improved. For the purposes of the present invention a therapeutic dose of antagonist is defined as an amount of antagonist, which when administered to a mammal, more preferably to a primate, most preferably to a human, inhibits, more preferably prevents new blood formation at the sites of disease being treated. The therapeutic index is defined by the amounts of VE-cadherin antagonist required to have a therapeutic effect relative to the amount of VE-cadherin that will cause unacceptable toxicity. That toxicity which is unacceptable will be readily understood by one of ordinary skill in the art.

In one preferred embodiment of the present invention VEGFR antagonist exerts its beneficial effect on VE-cadherin antagonist toxicity at doses of VEGFR antagonist which have anti-angiogenic, and in the case of neoplasms, anti-tumor effects. In one preferred embodiment of the present invention the protective effect of the VEGFR antagonist is achieved when the VEGFR antagonist is administered in a dose effective in preventing angiogenesis, even if administered alone. Therefore, the present invention permits the combined administration of a therapeutic amount of VE-cadherin antagonist and VEGFR antagonist with acceptable levels of toxicity or, more preferably, no toxicity. Additionally, the present invention permits the use of more than one anti-angiogenesis agent to enhance the therapeutic effect, and allows higher doses of VE-cadherin antagonist that would otherwise be possible. Optionally, one or more than one VE-cadherin antagonist is combined with one or more than one VEGFR antagonist. According to one embodiment of the invention, VE-cadherin antibody is administered in an amount of about 100 μg and VEGFR is administered at about 800 μg. For example, when administered to mice at high doses (≧100 μg), BV13 dramatically increases vascular permeability in lung tissue, and to a lesser extent the heart. On the other hand, DC101 (800 μg) (or p1C11) treatment reduces vascular permeability in tumor tissues.

In one preferred embodiment of the present invention, the antagonists are antibodies. Optionally, the antibodies are of mouse, rat, human, rabbit, or other mammalian origin. In another preferred embodiment the antibodies are chimeric or humanized as described below. Optionally, any VE-cadherin antibody is used Suitable VE-cadherin antibodies include, but are not limited to, BV13, 10G4, BV6, BV9, and Cad-5. In one preferred embodiment the anti-VE-cadherin antibody BV13 is used. Similarly, optionally an antibody against any VEGFR, including but not limited to FLK-1/KDR, FLT-1, and FLT-4 is used, including but not limited to Anti-FLT-1 mAb, DC101 (described in detail in U.S. Pat. No. 5,861,499 which is incorporated in its entirety herein by reference), and in one preferred embodiment, p1C11 mAb. Optionally, the VE-cadherin antagonist and VEGFR antagonist are administered sequentially, simultaneously or, more preferably, VEGFR antagonist is administered from about 2 to about 12 hours before VE-cadherin antagonist. Optionally the antagonists are administered intermittently or continuously. The antagonists are administered systemically, regionally or locally. Routes of administration include but are not limited to intravenously, intra-arterially, intraperitoneally, orally, sublingually, rectally, subcutaneously, intradermally, percutaneously, transcutaneously and intrathecally. One of ordinary skill in the art would be able to determine the optimal timing of antagonist administration and route of administration, optionally based on such factors as the disease location, the type of the disease, and the nature and properties of the antagonist(s). Appropriate doses of antagonist for use in other animals including larger mammals, including but not limited to humans can be readily determined by one of ordinary skill in the art, in view of the assays described herein, animal studies also described herein, and based on factors that include but are not limited to mass, body surface area, age and the medical condition of patients.

The present invention includes, but is not limited to, the use of one or more VE-cadherin antagonist together with one or more VEGFR antagonists together with other modalities of therapy to treat the disease of interest. Such diseases include but are not limited to neoplastic diseases, including solid tumors, and autoimmune and collagen vascular diseases such as rheumatoid arthritis and age-related macular degeneration. Such other treatments include, but are not limited to anti-inflammatory agents, chemotherapeutic agents and biologic response modifiers. While one preferred embodiment of the invention comprises antagonists that are antibodies, other embodiments include, but are not limited to a mixture of antagonists such that different types of antagonists are used. By way of illustration which is not intended to limit the scope of the invention, a combination of an antibody and antibody fragment or an antibody and small molecule or any other such combination of antagonist molecules described herein is encompassed by the scope of the present invention.

Antagonists to VE-cadherin and to VEGFR are either known in the art or are identified using assays that are known in the art. Selected antagonists are tested for use in the practice of the present invention using assays that evaluate the ability of one or more VEGFR antagonist selected for study to inhibit the toxicity of the VE-cadherin antagonist being studied, and optionally evaluate the ability of VE-cadherin antagonists to inhibit angiogenesis. Various combinations can be studied and optimal doses can be determined for further study, including clinical studies.

### In vivo Quantitative Permeability Assay

Vascular permeability in tissues is analyzed by a Miles-type assay with minor modification, as described below,. In brief, the test antagonist is administered by an appropriate route to mice (Balb/c, Jackson Laboratory, ME), optionally to other mammals, at various doses In a mouse, suitable doses for antibodies are 25-1000 μg/dose/200 μl PBS). Increased vascular permeability is determined by injecting Evans blue dye (100 μl/mouse of 1 μg/μl, Sigma) intraveneously at various times (1 h, 2 h, 7 h and 24 h) following antagonist administration. Twenty minutes later, the animals, optionally mice, are anesthetized optionally by ketamine and perfused with ˜20 ml of PBS via right ventricle. The organs are removed and homogenized in TCA/ethanol (1:1 v/v, 0.5 ml/tissue/eppendorf tube). The Evans blue content in the tissue homogenates is quantified by spectrophotometry (OD=510 nm). The antagonist effect on vascular permeability is measured as the percentage increase in Evans blue dye extravasation compared to a control animal. It will be apparent to one of ordinary skill in the art that the assay can be modified readily to permit the testing of various combinations and dosages of antagonist.

### Mouse Miles Assay

The assay is performed as described in Zebrowski et al, Vascular Endothelial Growth Factor Levels and Induction of Permeability in Malignant Pleural Effusions. Clin. Cancer Res. 5:3364-3368, (1999). In one preferred embodiment Balb/c mice are shaved on both sides of the flank and injected intravenously with 200 μl of Evans blue dye (0.5%) via the tail vein. Ten minutes later, mice are injected intradermally with 50 μl of saline or VEGF₁₆₅ (1 μg /μl). The subdermis is photographed 30 min later to record “leakage” of the dye into the dermal tissue. To test whether a VEGFR antagonist, by way of example which is not intended to limiting in any way, anti-FLK-1 mAb (DC101) can block the permeability effect of VEGF, the VEGFR antagonist under study, for example mAb DC101 (800 μg/dose) is administered to the mouse prior to the Evans blue dye injection. The route and dose of the VEGFR antagonist is determined by the properties of the particular antagonist and the intended route of administration in future studies. Preferably, the antagonist is administered at a dose or doses that have anti-angiogenic properties. The present assay permits the evaluation of various types of antagonists at various doses, routes of administration and schedules, as is readily apparent to one of ordinary skill in the art.

The antagonists of the present invention comprise, for example, recombinant polypeptide, natural polypeptide or synthetic polypeptide. Preferably recombinant polypeptide is used. The present invention also comprises nucleic acid molecules, including DNA, RNA, DNA/RNA hybrids which encode the antagonists of the present invention. The polypeptides and polynucleotides of the present invention are preferably provided in an isolated form, and preferably are purified to homogeneity.

The polynucleotide encompassed within the scope of this invention include polynucleotides encoding for a soluble form of the VE-cadherin polypeptide, which is the extracellular portion of the polypeptide. Fragments of the polynucleotides of this invention are used for example as a hybridization probe for a cDNA library to isolate the full length cDNA of VE-cadherin protein or to isolate other cDNAs which have a high sequence similarity to the gene or similar biological activity, using methods that are known in the art. The present invention also relates to vectors and host cells that are genetically engineered (transduced or transformed or transfected).

The constructs in host cells are used in a conventional manner to produce the gene product encoded by the recombinant sequence. Alternatively, the polypeptides of the invention are synthetically produced by conventional peptide synthesizers.

Mature proteins are expressed in mammalian cells, yeast, bacteria, or other cells under the control of appropriate promoters. Cell-free translation systems are also employed to produce such proteins using RNA derived from the DNA constructs of the present invention. Appropriate cloning and expression vectors for use with prokaryotic and eukaryotic hosts are described by Sambrook, et al., Molecular Cloning: A Laboratory Manual, Second Edition, Cold Spring Harbor, N.Y., (1989), the disclosure of which is hereby incorporated by reference.

Various mammalian cell culture systems can also be employed to express recombinant protein. Examples of mammalian expression systems include the COS-7 lines of monkey kidney fibroblasts, described by Gluzman, Cell, 23:175 (1981), and other cell lines capable of expressing a compatible vector, for example, the C127, 3T3, CHO, HeLa and BHK cell lines.

According to another embodiment of the invention, the polynucleotides and polypeptides of the present invention are employed as research reagents and materials for discovery of treatments and diagnostics for human diseases.

A small molecule which binds to a VE-cadherin peptide and makes it inaccessible to ligands, for example small peptides, peptide-like molecules, or non-peptide based molecules are also encompassed within the scope of this invention. Inhibitors of tyrosine kinase are among many small molecules that are intended.

According to a general embodiment of the invention disclosed herein, recombinant, mammalian and human VE-cadherin proteins are cloned and expressed to generate polyclonal and monoclonal antibodies against VE-cadherin. Transfectant cell lines expressing mouse or human full-length VE-cadherin are established to test reactivity of anti-VE-cadherin mAb and to establish in vitro functional assays. Transfectant cell lines are also generated that express various regions of the VE-cadherin extracellular domain to facilitate mapping of VE-cadherin mAb binding determinants.

These antibodies are, for example, polyclonal or monoclonal antibodies. The present invention also includes chimeric, single chain, and humanized antibodies, as well as Fab fragments, or the product of an Fab expression library. Various procedures known in the art may be used for the production of such antibodies and fragments.

Techniques used for preparation of monoclonal antibodies, include but are not limited to, the hybridoma technique (Kohler & Milstein, Nature, 256:495-497 (1975)), the trioma technique, the human B-cell hybridoma technique (Kozbor et al., Immunology Today 4:72, (1983)), and the EBV-hybridoma technique to produce human monoclonal antibodies (Cole, et al., 1985, In Monoclonal Antibodies and Cancer Therapy, Alan R. Liss, Inc., pp. 77-96).

Techniques described for the production of single chain antibodies (U.S. Pat. No. 4,946,778, incorporated herein by reference) are adapted to produce single chain antibodies to immunogenic polypeptide products of this invention. Also, transgenic mice are used to express humanized antibodies to immunogenic polypeptide products of this invention.

In one strategy, the human heavy and light chain immunoglobulin gene complexes are introduced into a mouse germ line to yield animals whose antibody production is purely human.

Epitope binding components of the present invention refer to proteins consisting of one or more polypeptides substantially encoded by genes of the immunoglobulin superfamily (e.g., see The Immunoglobulin Gene Superfamily, A. F. Williams and A. N. Barclay, in Immunoglobulin Genes, T. Honjo, F. W. Alt, and T. H. Rabbitts, eds., (1989) Academic Press: San Diego, Calif., pp.361-387, which is incorporated herein by reference). For example, an epitope binding component may comprise part or all of a heavy chain and part or all of a light chain, or may comprise only part or all of a heavy chain. However, an epitope binding component must contain a sufficient portion of an immunoglobulin superfamily gene product so as to retain the property of binding to a specific target, or epitope.

Included within the scope of this invention are bispecific antibodies that are formed by joining two epitope binding components that have different binding specificities. In preferred embodiments of the invention, the epitope binding component is encoded by immunoglobulin genes that are “chimeric” or “humanized” (see generally, Queen (1991) Nature 351:501, which is incorporated herein by reference).

VE-cadherin antibodies, epitope binding components, their dimers, or individual light and heavy chains are purified according to standard procedures of the art, including ammonium sulfate precipitation, fraction column chromatography, gel electrophoresis and the like (see generally, Scopes, R., Protein Purification, Springer-Verlag, N.Y. (1982)). Once purified, partially or to homogeneity as desired, the antibodies and fragments thereof are then used therapeutically or in developing and performing assay procedures, immunofluorescent stainings, and the like (see generally, Immunological Methods, Vols. I and II, Lefkovits and Pernis, Eds., Academic Press, New York, N.Y. (1979 and 1981)).

Pharmaceutical compositions comprising the antagonists of the present invention are encompassed by the present invention. Actual methods for preparing administrable compositions will be known or apparent to those skilled in the art and are described in more detail in, for example, Remington's Pharmaceutical Science, 15th Ed., Mack Publishing Company, Easton, Pa. (1980), which is incorporated herein by reference.

Another aspect of the present invention is the use of a VEGFR antagonist, such as is disclosed herein to treat disease states wherein there is increased vascular permeability/pathological vascular permeability. Such conditions include but are not limited to “leaky capillary syndrome” including but not limited to adult respiratory distress syndrome (ARDS), drug related conditions and edema states. Mammals, more preferably primates, most preferably humans with such illnesses are treated with a therapeutic amount of VEGFR antagonist in order to improve or cure the illness. Appropriate methods of administration and dosages will be readily apparent to those of ordinary skill in the art.

The invention will be more fully understood by reference to the following examples. These examples are not to be construed in any way as limiting the scope of this invention. All literature cited herein is specifically incorporated by reference.

### EXAMPLES

### Example 1

### VE-Cadherin Mab Inhibition of Endothelial Cell Tube Formation

Monoclonal antibodies (Cad5, BV9, BV6, TEA, and Hec 1.2) specific for different regions of human VE-cadherin and mAb BV13 specific for mouse are tested for their ability to inhibit the formation of tubes. The tube-forming assay is established to specifically identify VE-cadherin antagonists with junction formation inhibiting activity. The assay is performed in a collagen sandwich culture as described by Bach, et al., Experimental Cell Research, 238: 324-344 (1998). Type I collagen from rat tails (250 μl of 1 μg/μl, neutral pH) is plated onto each well of 24-well culture plates and incubated at 37° C. until a collagen gel is formed. Human umbilical vein endothelial cells (HUVEC) or H5V mouse endothelioma cells are seeded on the collagen-coated wells and monolayers established by incubation for 24 h at 37° C. Unattached cells are then aspirated and overlaying collagen gels generated using the same procedure. Various amounts of test antibodies (5-10 μg/μl) are added into the culture medium as well as the collagen gels. Complete tubular structures are detected and examined in 34 days (verified by thin sectioning and electron microscopy). Anti-human VE-cadherin mAb Cad 5, BV9, BV6 and anti-mouse VE-cadherin mAb BV13 dramatically inhibit tube formation to a similar extent at a concentration of 5-10 μg/μl.

### Example 2

### Anti-murine VE-cadherin Antibody (BV13) Inhibits Angiogenesis in Vivo

To demonstrate that anti-mouse VE-cadherin antibody can directly inhibit angiogenesis in vivo, BV13 is tested in three models, i.e., corneal micropocket and alginate-encapsulated tumor cell (Plunkett, M. & Hailey, J., Lab. Invest. 62:510-517 (1990)) and Matrigel plug (Passaniti, et al., Lab. Invest. 67: 519-528 (1992)) assays. In all three models, BV13 shows significant inhibition of angiogenesis. The models are as follow:

(I) Corneal Micropocket Model: The assay was performed with modifications as described by Kenyon, et al., Invest. Ophthalmol, Vis. Sci. 37: 1625-1632 (1996). A corneal micropocket was created in both eyes of 4-6 week old Balb/c mice (Jackson Laboratory, Bar Harbor, Me.) with a modified von Graefe cataract knife. One 0.34×0.34-mm sucrose aluminum sulfate (Bukh Meditec, Copenhagen, Denmark) pellet coated with hydron polymer type NCC (IFN Sciences, New Brunswick, N.J.), containing 50 ng of bFGF and 1 μg of mAb BV13 or control rat IgG1, is implanted into each pocket 1 mm away from the limbic vessels. The corneas are routinely examined and photographed by slit-lamp biomicroscope on postoperative days 5-7. Maximum capillary formation was observed in eyes with the bFGF and control rat IgG1 pellets on day 6 after implantation. Each experimental group contained 6 mice. All the eyes showed comparable vascularization within the same experimental group. The effect on bFGF-induced neovascularization was quantified based on the vessel density and vessel length. The eyes of the animals receiving BV13 treatment were found to have much fewer and thinner corneal limbic capillaries (>70% inhibition) than those of animals receiving a rat IgG1 control antibody.

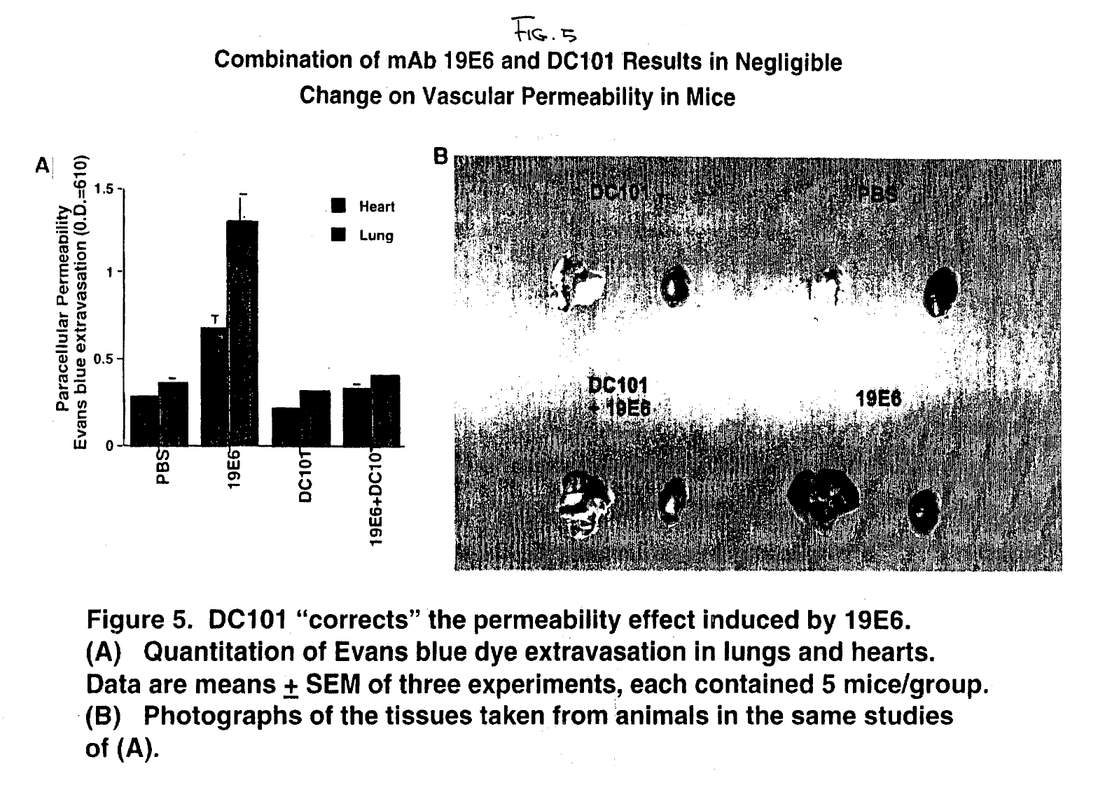

(II) Alginate-Encapsulated Tumor Cell Assay: The effect of BV13 mAb is also evaluated in an alginate-encapsulated tumor cell assay as described previously by Plunkett, M. & Hailey, J. A., Lab. Invest. 62:510-517 (1990). Alginate beads (4 beads/mouse) containing 5×10⁴ Lewis Lung tumor cells per bead are surgically implanted subcutaneously into the back of C57BL/6 mice. Growth factors produced by the encapsulated tumor cells induced vascularization of the beads over 12 days, which is then measured by injecting mice intravenously with FITC-dextran prior to sacrifice and measuring uptake of FITC-dextran into beads. BV13 administered systemically by intraperitoneal (i.p.) injection, 25 μg/dose, every 3 days significantly reduced (>50% at 25 μg/dose) the results of the vascularization of alginate beads compared to mice injected with control antibody, as shown in FIG. 5.

(III) Matrigel Plug Assay: The potent anti-angiogenic effect of BV13 is also confirmed in a modified Matrigel plug model (Passaniti, et al., Lab. Invest. 67:519-528 (1992). Matrigel supplemented with bFGF and VEGF is injected subcutaneously into C57BL/6 mice. After 21 days, extensive vascularization is observed throughout the plugs. Plugs taken from mice treated with 25 or 50 μg/dose of BV13 antibody had markedly reduced vascularization of plugs as examined by vessel staining with anti-von Willebrand Factor antibody. Matrigel plug angiogenesis is quantified by injecting mice with FITC-dextran and measuring the uptake of FITC-dextran into plugs after their removal from mice (Prewett, et al., Cancer Res. 59:5209-5218 (1999)). BV13 treatment even at a lower dose (25 μg/dose) significantly inhibited (approximately 80%; p<0.01) the amount of FITC-dextran in Matrigel plugs compared to control antibody.

### Example 3

### Anti-VE-cadherin Mab BV13 Inhibits Tumor Growth and Metastasis

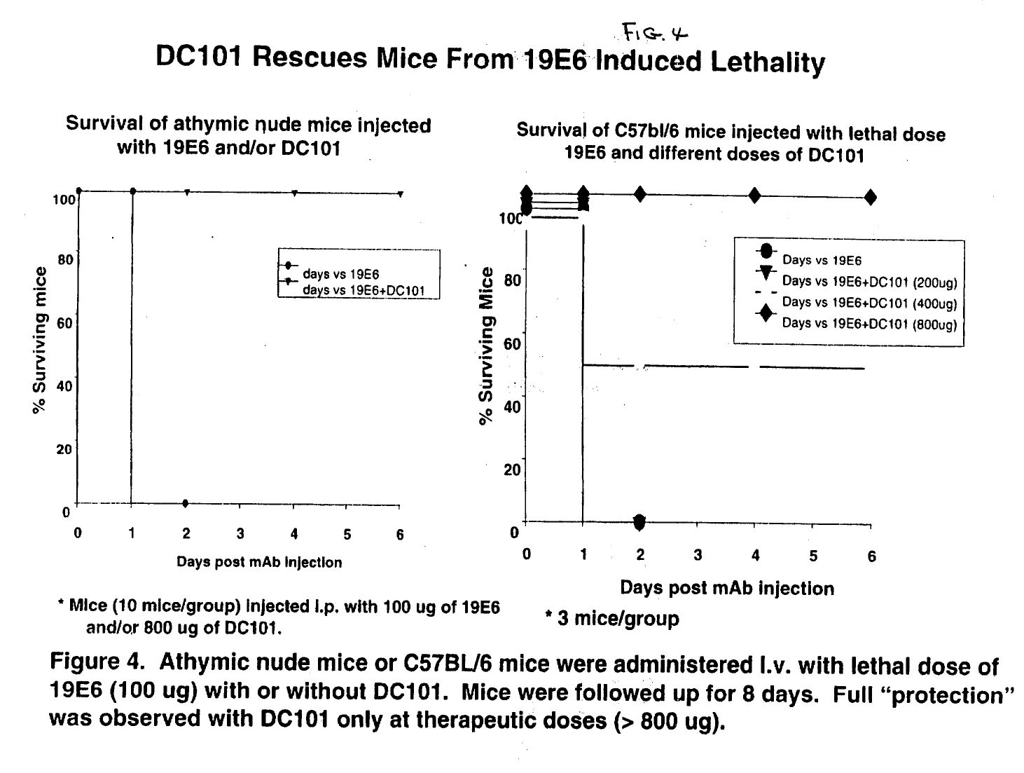

To examine the anti-tumor effect of VE-cadherin, antibody mAb BV13 is tested in Lewis lung animal models. BV13 treatment of mice with subcutaneous Lewis lung tumors inhibited tumor growth in a dose-dependent manner compared to control antibody (see FIG. 4). In the Lewis lung metastasis model, BV13 treatment significantly inhibits the growth of pulmonary metastases. Lung weight (80%) and metastases (>90%) are dramatically reduced in BV13-treated mice compared to control group. In fact, 10 out of 10 mice in the BV13-treated group showed no macroscopic evidence of pulmonary metastases, whereas all mice in the control group showed large numbers of lung metastases. The effect of mAb BV13 therapy on human tumor growth is also examined in A431 xenograft model. Significant inhibition of tumor growth is observed with 50 μg/dose of BV13 without complete tumor regression. Tumors derived from BV13-treated mice showed a marked decrease in cellularity and areas of necrosis replaced by fibrous tissue.

### Example 4

### Anti-VE-cadherin Mab BV13 Increases Lung Vascular Permeability

Systemic treatment of mice with anti-VE-cadherin mAb BV13 at doses of 100 μg resulted in increased vascular permeability in the lungs (>150%) and to a lesser extent in the heart tissue (approx. 75%). The permeability effects of BV13 treatment are manifested by labored respiration and mortality in a large percentage of treated mice. By electron microscopy studies, interstitial edema and accumulation of mixed type of inflammatory cells in hearts and lungs from the BV13-treated mice are observed. In these mice, alveolar capillaries and some large pulmonary vessels showed loosely organized endothelium and denuded basement membrane, as observed by confocal examination of the injected dye (roaming-labeled Ricinus Communis I Pectin). The in vivo effects of mAb BV13 on vascular permeability are described in Corada, et al., Proc. Natl. Acad. Sci. 96:9815-9820 (1999) incorporated by its entirety herein.

### Example 5

### Cell Permeability Assay

The cell permeability assay is performed by seeding VE-cadherin-expressing CHO cell transfectants or endothelial cells in the top chamber of Costar Transwells (Coming Costar Corp., Cambridge, Mass.). Cultures are incubated for 2 days to allow for formation of adherens junctions and a confluent cell monolayer. Test antibodies are then added to the top chamber of cells along with FITC-dextran. The anti-VE-cadherin antibody effect on cell permeability (junction disruption) is measured as a function of FITC-dextran that permeates into the bottom chamber. Several anti-VE-cadherin antibodies are tested in this assay and showed distinct effects on cellular permeability. Antibodies (Cad5, BV9 and BV13) exhibited significant effect (>100% increase on permeability) whereas BV6 showed a less dramatic effect (approximately 40% increase in permeability). Antibodies TEA and Hec1.2 do not appear to exert any significant effect on cell permeability.

### Example 6

### Junction Formation Assay

The junction formation assay is developed based on a modification of the calcium switch assay (Gumbiner, B., & Simons, K., J. Cell Biol. 102:457-468 (1986)). Transfectant CHO cells or endothelial cells expressing VE-cadherin are plated onto glass slides and allowed to form a confluent monolayer. The adherens junctions of the monolayer are artificially disrupted by depleting calcium from the culture medium by incubation with 5 mM EGTA for 30 min. EGTA-containing media is then removed and fresh media containing calcium is added to the culture to allow for formation of adherens junctions. The inhibition of junction formation is measured by addition of various concentrations of anti-VE-cadherin mAb at the time calcium-containing fresh media is added. The kinetics of junction disruption and junction reformation processes correlate with the disappearance and reappearance of VE-cadherin in the adherens junctions. The formation of adherens junctions is visualized by immunofluorescent staining with a polyclonal antibody specific for mouse or human V-cadherin. Immunostaining on another junctional adhesion molecule (CD31) is routinely included to ensure that the treated cell monolayer does not retract. The effect of antibodies on the reformation of the disrupted junctions is observed by visualization of VE-cadherin staining at the cell-to-cell contacts and further quantified by performing the assay in Costar transwells using cell permeability as a readout.

### Example 7

### Test Candidate Mab for Effects on Vascular Permeability in Vivo

Vascular permeability in tissues are analyzed by a Miles-type assay with some modification (Corada, et al., Proc. Natl. Acad Sci. 96:9815-9820 (1999)). In brief, the test mAb are administered either intraperitoneally or intravenously to mice at various doses (50-1000 μg/dose). Increased vascular permeability are determined by injecting Evans blue dye (100 μl of 1 mg/ml) intravenously at various times (6 h, 12 h, 24 h and 48 h) following mAb administration. Twenty minutes later, mice are anesthetized by ketamine and perfused with approximately 20 ml of PBS. Mouse organs are removed and homogenized in TCA/ethanol (1:1 v/v). The Evans blue content in the tissue homogenates are quantified by spectrophotometry (OD=510 nm). The mAb effect on vascular permeability is measured as the percentage increase in Evans blue dye compared to control antibody. The effect of new VE-cadherin mAb are compared to anti-VE-cadherin mAb BV13, which typically results in 150-200% increase in permeability. Tissues from anti-VE-cadherin mAb-treated mice are also examined by histology for evidence of pathology. Those mAb which have no effect on vascular permeability in normal tissues are further tested in angiogenesis and tumor models.

### Example 8

### Antibody Combination Therapy

Monoclonal antibodies against VE-cadherin (BV13, also called BV13) and VEGFR-2 (DC101) are developed and characterized as described previously ( see, Corada et al., Proc. Natl. Acad. Sci. 96:9815-9820 (1999), and Prewett et al., Cancer Res. 59: 5209-5218 (1999), both incorporated herein by reference). Hybridoma cells are cultured in serum-free medium and antibodies are purified from conditioned media by protein G-Sepharose chromatography. All antibody preparations used in animal studies, including control rat IgG (Jackson ImmunoResearch Laboratories, West Grove, Pa.), contained<1.25 EU/ml of endotoxin as assessed by the Limulus Amebocyte Lysate assay kit (BioWhittaker, Walkersville, Md.). Nude mice or C57B16 mice (Harlan Sprague Dawley, Inc., Indianapolis, Ind.) are injected intraperitoneally with 100 ug of BV13/mouse. Mice are then randomly divided into several groups (3 mice/group). Each receives either BV13 alone, or BV13 plus DC101 (various doses of DC101, 200, 400, 600 and 800 μg/mouse, intraperitoneally). Mice are housed in pathogen-free environment and are followed up for one week with routine check every day for lethality and signs of sickness.

### Example 9

### In vivo quantitative Permeability Assay

Vascular permeability in tissues is analyzed by a Miles type assay with minor modification. In brief, the test mAb are administered either intraperitoneally or intravenously to mice (Balb/c, Jackson Laboratory, Me.) at various doses (25-100 mg/dose/200 mlPBS). Increased vascular permeability is determined by injecting Evans blue dye (100 ml/mouse of 1 mg/ml, Sigma) intravenously at various times (1 h, 2 h, 7 h and 24 h) following mAb administration. Twenty minutes later, mice are anesthetized by ketamine and perfused with ˜20 ml of PBS via right ventricle. Mouse organs are removed and homogenized in TCA/ethanol (1:1 v/v, 0.5 ml/tissue/eppendorf tube). The Evans blue content in the tissue homogenates is quantified by spectrophotometry (OD=510 nm). The mAb effect on vascular permeability is measured as the percentage increase in Evans blue dye extravasation compared to control antibody.

### Example 10

### Mouse Miles Assay

The assay is performed as described in Zebrowski et al (1999) incorporated herein by reference. Balb/c mice are shaved on both sides of flank and injected intravenously with 200 μl of Evans blue dye (0.5%) via the tail vein. Ten minutes later, mice are injected intradermally with 50 μl of saline or VEGF₁₆₅ (1 μg/ml). The subdermis is photographed 30 min later to record “leakage” of the dye into the dermal tissue. To test whether anti-FLK-1 mAb (DC101) blocks the permeability effect by VEGF, mAb DC101 (800 μg/dose) is administered to mice intravenously 6 h prior to Evans blue dye injection.

We claim:

1. A method of inhibiting pathological angiogenesis in an animal in need thereof, comprising:
administering an antagonist of a vascular endothelial cadherin; and
administering an antagonist of a vascular endothelial growth factor receptor.

2. The method of claim 1, wherein the animal is a mammal.

3. The method of claim 2 wherein the mammal is a human.

4. The method of claim 1, wherein the vascular endothelial growth factor receptor is FLK-1, KDR, FLT-1 or FLT-4.

5. The method of claim 4, wherein the vascular endothelial growth factor is KDR.

6. The method of claim 1, wherein the antagonist of vascular endothelial growth factor receptor is selected from the group comprising, a protein, a peptide, a polypeptide, an antisense molecule, a small molecule, an antibody, and an antibody fragment.

7. The method of claim 1, wherein the vascular endothelial cadherin antagonist is selected from the group comprising, a protein, a peptide, a polypeptide, an antisense molecule, a small molecule, an antibody, and an antibody fragment.

8. The method of claim 7, wherein the antibody is selected from the group comprising BV13, 10G4, BV6, BV4, Cad-5, BV9, TEA, and Hec 1.2.

9. The method of claim 8, wherein the antibody is BV13.

10. The method of claim 1, wherein the antagonist of the vascular endothelial growth factor receptor interacts with a vascular endothelial growth factor receptor.

11. The method of claim 1, wherein the antagonist of the vascular endothelial growth factor receptor interacts with a vascular endothelial growth factor ligand.

12. The method of claim 6, wherein the antibody is selected from the group comprising p1C11 and DC101.

13. The method of claim 12, wherein the antibody is p1C11.

14. A method of modulating angiogenesis in an animal in need thereof, while reducing toxicity, comprising:
administering an antagonist of vascular endothelial cadherin; and
administering an antagonist of vascular endothelial growth factor receptor.

15. A method of treating a disease associated with pathological angiogenesis, comprising:
administering an antagonist of vascular endothelial cadherin; and
administering an antagonist of vascular endothelial growth factor receptor.

16. A method of treating, with reduced toxicity, a disease associated with pathological vascular permeability, comprising:
administering an antagonist of vascular endothelial cadherin; and
administering an antagonist of vascular endothelial growth factor receptor.

17. The method of claim 15, wherein the disease is selected from the group comprising a neoplastic disease, an autoimmune disease and a collagen vascular disease.

18. The method of claim 17, wherein the neoplastic disease is a solid tumor.

19. The method of claim 13, wherein the antagonist of the vascular endothelial cadherin is administered after the antagonist of the vascular endothelial growth factor receptor.

20. The method of claim 14, wherein the antagonist of the vascular endothelial growth factor receptor is administered after the antagonist of the vascular endothelial cadherin.

21. The method of claim 14, wherein the antagonist of the vascular endothelial growth factor receptor is administered at substantially the same time as the antagonist of the vascular endothelial cadherin is administered.

22. A composition for modulating angiogenesis in an animal in need thereof, while reducing toxicity, comprising:
an antagonist of a vascular endothelial cadherin; and
an antagonist of a vascular endothelial growth factor receptor.

23. The composition of claim 22, wherein the vascular endothelial growth factor receptor is FLK-1, KDR, FLT-1 or FLT-4.

24. The composition of claim 23 wherein the vascular endothelial growth factor is KDR.

25. The composition of claim 22 wherein the antagonist of vascular endothelial cadherin is selected from the group comprising, a protein, a peptide, a polypeptide, an antisense molecule, a small molecule, an antibody, or an antibody fragment.

26. The composition of claim 22, wherein the vascular endothelial cadherin antagonist is selected from the group comprising, a protein, a peptide, a polypeptide, an antisense molecule, a small molecule, an antibody, and an antibody fragment.

27. The composition of claim 26, wherein the antibody is selected from the group comprising BV13, 10G4, BV6, BV4, Cad-5, BV9, TEA, and Hec.1.2.

28. The composition of claim 27, wherein the antibody is BV13.

29. The composition of claim 22, wherein the antagonist of vascular endothelial growth factor receptor is selected from the group comprising, a protein, a peptide, a polypeptide, an antisense molecule, a small molecule, an antibody, and an antibody fragment.

30. The composition of claim 29, wherein the antibody is p1C11.

31. The composition of claim 22, wherein the animal is a mammal.

32. The composition of claim 31, wherein the mammal is a human.

33. The composition of claim 22, wherein the disease is selected from the group comprising a neoplastic disease, an autoimmune disease and a collagen vascular disease.

34. A method of modulating angiogenesis in an animal in need thereof, while reducing toxicity, comprising:
administering p1C11; and
administering BV13.

35. A method of inhibiting the growth of a solid tumor, comprising:
administering an antagonist of a vascular endothelial cadherin; and
administering an antagonist of a vascular endothelial growth factor receptor, wherein the development of pathologically increased vascular permeability is inhibited.

36. A method of inhibiting pathologically increased vascular permeability to an animal in need thereof, comprising:
administering an antagonist of a vascular endothelial growth factor receptor.

37. The method of claim 36, wherein the vascular endothelial growth factor receptor is FLK-1, KDR, FLT-1 or FLT-4.

38. The method of claim 37, wherein the vascular endothelial growth factor receptor is KDR.

39. The method of claim 36, wherein the antagonist of the vascular endothelial growth factor receptor is selected from the group comprising, a protein, a peptide, a polypeptide, an antisense molecule, a small molecule, an antibody, and an antibody fragment.

40. The method of claim 39, wherein the antagonist of the vascular endothelial growth factor receptor is an antibody.

41. The method of claim 40, wherein the antibody is p1C11.

42. The method of claim 36, wherein the pathologically increased vascular permeability is a result of adult respiratory distress syndrome or a pediatric respiratory distress syndrome.

43. A composition for inhibiting pathologically increased vascular permeability in an animal in need thereof, comprising:
an antagonist of a vascular endothelial growth factor receptor.

44. The composition of claim 43, wherein the vascular endothelial growth factor receptor is FLK-1, KDR, FLT-1 or FLT-4.

45. The composition of claim 44, wherein the vascular endothelial growth factor receptor is KDR.

46. The composition of claim 43, wherein the antagonist of the vascular endothelial growth factor receptor is selected from the group comprising, a protein, a peptide, a polypeptide, an antisense molecule, a small molecule, an antibody, and an antibody fragment.

47. The composition of claim 46, wherein the antagonist of the vascular endothelial growth factor receptor is an antibody.

48. The composition of claim 47, wherein the antibody is selected from the group comprising p1C11 and DC101.

49. The composition of claim 48, wherein the antibody is p1C11.
